(19) United States
(12) Patent Application Publication
(10) Pub. No.: US 2013/0104909 A1
(43) Pub. Date: May 2, 2013

(54) Surgical Drape

(71) Applicant: The Curators of the University of Missouri, Columbia, MO (US)

(72) Inventors: Breton Foster Barrier, Columbia, MO (US); Michael Wayne McCullough, Columbia, MO (US)

(73) Assignee: The Curators of the University of Missouri, Columbia, MO (US)

(21) Appl. No.: 13/666,351

(22) Filed: Nov. 1, 2012

Related U.S. Application Data
(60) Provisional application No. 61/628,507, filed on Nov. 1, 2011.

(51) Int. Cl. A61B 19/10

(52) U.S. Cl. 128/852

(57) ABSTRACT

The present disclosure provides a surgical drape for providing a sterile field around the vaginal area of a patient during vaginal surgical procedures. In various embodiments the drape comprises a main body and an access opening centrally disposed within the main body. The drape additionally includes a plurality of pleats disposed along opposing lateral side portions of main body. The pleats are structured and operable to allow the drape to be contoured around the patient's abdomen, pelvis and legs, and to provide stability to the drape such that the drape will not be easily displaced from the patient. Furthermore, the drape includes a plurality of equipment retention devices disposed on the main body that are structured and operable to removably retain surgical equipment during the respective surgical procedure.

### CROSS-REFERENCE TO RELATED APPLICATIONS

This application claims the benefit of U.S. Provisional Application No. 61/628,507, filed on Nov. 1, 2011. The disclosure of the above application is incorporated herein by reference in its entirety.

### FIELD

The present teachings relate to a surgical drape, more specifically, to a surgical drape that includes a plurality of surgical equipment retention devices structured and operable to assist in episiotomy or obstetrical laceration repair following vaginal delivery or other vaginal surgery.

### BACKGROUND

The statements in this section merely provide background information related to the present disclosure and may not constitute prior art.

Typically, during an obstetrical laceration or episiotomy repair following vaginal delivery or other vaginal surgery, the medical staff or physician will dispose a plurality of sterile drapes over the patient's abdomen, pelvis and legs to provide a sterile field around the vaginal area while still allowing surgical access to the vaginal area. However, when disposed in this way common surgical drapes tend to slide or drop from the patient during the procedure. Moreover, if the physician sets equipment used during the respective procedure, e.g., hemostats, forceps, sutures, gauze, sponges, and other surgical instruments, tools and supplies, on top of the drapes during the procedure, due to the weight of the equipment and the contour of the underlying surface, i.e., the patient's body, the equipment will have a tendency to slide off the drapes and/or displace the drapes from the desired position and orientation. This can complicate and disrupt the procedure and cause possible contamination of the equipment and/or the physician's hands.

### SUMMARY

The present disclosure provides a surgical drape for providing a sterile field around the vaginal area of a patient during vaginal surgical procedures. In various embodiments the drape comprises a main body having an access opening centrally disposed therein and a plurality of pleats disposed along opposing lateral side portions of main body. The pleats are structured and operable to allow the drape to be contoured around the patient's abdomen, pelvis and legs, and to provide stability to the drape such that the drape will not be easily displaced from the patient. The drape additionally includes a plurality of equipment retention devices disposed on the main body that are structured and operable to removably retain surgical equipment during the respective surgical procedure.

Further areas of applicability of the present teachings will become apparent from the description provided herein. It should be understood that the description and specific examples are intended for purposes of illustration only and are not intended to limit the scope of the present teachings.

### DRAWINGS

The drawings described herein are for illustration purposes only and are not intended to limit the scope of the present teachings in any way.

FIG. 1 is an illustration of a surgical drape for providing a sterile field around the vaginal area of a patient during vaginal surgery procedures, in accordance with various embodiments of the present disclosure.

FIG. 2 is an illustration of the drape shown in FIG. 1 disposed and positioned on a mannequin illustrating the contourability and functionality of the various features of the drape, in accordance with various embodiments of the present disclosure. For modesty, dignity and discretion purposes, the interior of an access opening of the drape shown in FIG. 2 has been concealed.

FIG. 3A is an illustration of a back side of a top portion of the drape shown in FIG. 1 illustrating a drape retention device of the drape, in accordance with various embodiments of the present disclosure.

FIG. 3B is an illustration of a front side top portion of the drape shown in FIG. 1 illustrating the drape retention device, in accordance with various other embodiments of the present disclosure.

FIG. 4 is an illustration of a center portion of a back side of the drape shown in FIG. 1, illustrating an access opening of the drape having a reinforcement frame and adhesive strip disposed around the access opening, in accordance with various embodiments of the present disclosure.

FIG. 4A is an illustration of the center portion of the back side of the drape shown in FIG. 1, illustrating the access opening of the drape having a combined reinforcement frame and adhesive strip disposed around the access opening, in accordance with various embodiments of the present disclosure.

FIG. 5A is an illustration of a suture retraction of the drape shown in FIG. 1, in accordance with various embodiments of the present disclosure.

FIG. 5B is an illustration of the suture retraction of the drape shown in FIG. 1, in accordance with various other embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

Unless otherwise defined, all technical and scientific terms used herein have the same meaning as commonly understood by one of ordinary skill in the art to which this invention belongs. All publications, patent application, patents, and other references mentioned herein are incorporated by reference in their entirety.

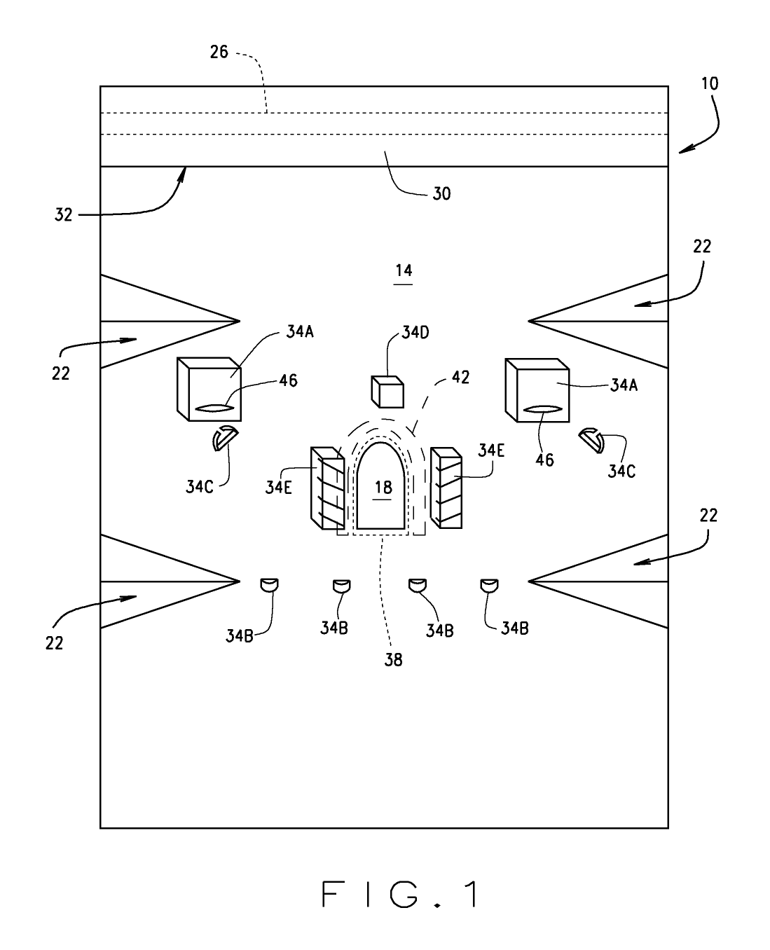

Referring to FIG. 1, the present disclosure provides a surgical drape 10 for providing a sterile field around the vaginal area of a patient during obstetrical laceration repair procedures, episiotomy repair procedures following vaginal delivery, and other vaginal surgical procedures. As described herein, the drape 10 is structured and operable to contour to the patient's body such that the drape 10 can be stably disposed on and across the patient's abdomen, pelvis, upper legs, and vaginal area, while providing surgical access to the vaginal area. More specifically, the drape 10 is structured and operable to be placed and positioned on the patient such that the drape 10 will not readily move, slide, fall off, or otherwise be easily displaced.

The drape 10 generally comprises a main body 14, an access opening 18 disposed generally in the center of the main body 14 and a plurality of pleats 22 formed, or disposed, along opposing lateral side or edge portions of the main body 14. The main body 14 is constructed of any cloth and/or paper type material that is well known in the art for the construction of known surgical drapes, covers, gowns, hats, shoe covers, etc., or any other suitable cloth-like material that can provide a sterile field around vaginal area. For example, in various embodiments, the entire drape 10, or at least the main body 14, is constructed of a disposable paper type material that is well known in the art for the construction of known surgical drapes, covers, gowns, hats, shoe covers, etc.

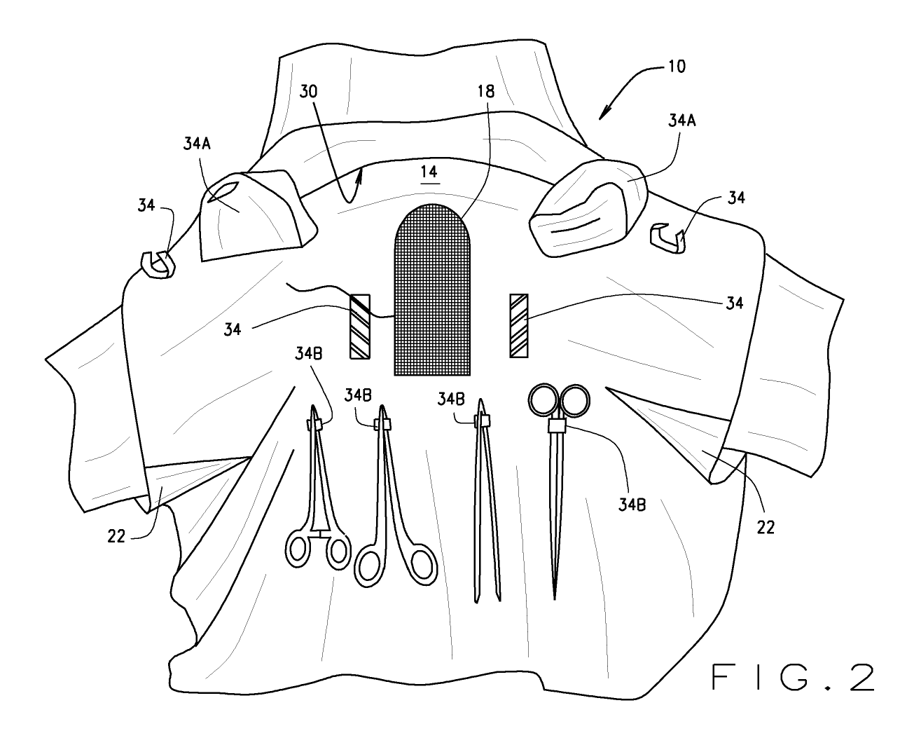

The access opening 18 is structured and operable to provide access the patient's vaginal area, and can have any size and shape suitable to provide access to the patient's vaginal area as desired by the respective physician. The pleats 22 are structured and operable to allow the drape 10 to be wrapped, or contoured, around the patient's abdomen, pelvis and legs, as illustrated in FIG. 2. In various embodiments, the drape 10 can additionally include a drape retention device 26 formed or disposed along a top portion of the drape 10. The drape retention device 26 is structured and operable to further wrap or contour the drape 10 around the patient's abdomen, pelvis and legs, and also to retain the disposition and placement of the drape 10 on the patient so that the drape will not readily move, slide, fall off the patient, or otherwise be easily displaced.

In various embodiments, the drape 10 can further include a disposition flap 30 that forms an elongated pocket 32 along a top edge portion of the drape 10. The elongated pocket 32 is structured and operable to provide a sterile means for the physician (or other medical team member) to handle, dispose and position the drape 10 on the patient, as described below. Furthermore, in various embodiments, the drape 10 can further include a plurality of equipment retention devices 34A, 34B, 34C, 34D and 34E (cumulatively referred to herein as equipment retention devices 34) that are structured and operable to provide the respective physician with multiple means for the placement and retention of various types of surgical equipment, e.g., forceps, hemostats, sutures, gauze, sponges, and other surgical instruments, tools and supplies, during the respective procedure. The equipment retention devices 34 are formed, connected to and/or disposed on the main body 14 around the access opening 18 so as to be conveniently, efficiently and ergonomically located and accessible when the drape 10 is positioned on a patient, as described below.

Still further, in various embodiments, the drape 10 can further includes a reinforcement frame 38 disposed around at least a portion of the access opening 18. The reinforcement frame 38 is structured and operable to provide integrity and support to the access opening 18 such that the access opening 18 will remain open and not collapse or otherwise close during use of the drape 10. Still yet further, in various embodiments, the drape 10 can further include at least one adhesive strip 42 disposed on a back side of the drape 10 around at least a portion of the access opening 18 for removably adhering a periphery of the access opening 18 to the patient, thereby maintaining the drape 10 in place and providing interference free access to the vaginal area by the physician.

Referring now to FIGS. 1 and 2, as described above, in various embodiments, the drape 10 includes a plurality of pleats 22 formed, or disposed, along opposing lateral side or edge portions of the main body 14 that are structured and operable to allow the drape 10 to be wrapped and contoured around the patient's abdomen, pelvis and legs. That is, the pleats 22 allow the drape 10 to be laid across the patients abdomen, pelvis and legs then pressed down around the patient's pelvis, hips and upper thighs and tucked under the patient's buttocks and/or hamstrings such that the drape 10 is contoured around the patient's abdomen, pelvis and legs, as illustrated in FIG. 2. Moreover, contouring the drape 10 around the patient's abdomen, pelvis and legs, via the pleats 22, provides stability to the drape 10 such that the drape 10 will not readily move, slide, fall off the patient, or otherwise be easily displaced. More specifically, when the pleats 22 are utilized to contour the drape 10 around the patient's abdomen, pelvis and legs, the drape 10 makes contact with the surface area of the patient's inner, upper and outer thighs, along with the patient's abdomen, hips and buttocks. Such surface contact area is considerably larger than that of known drapes, and moreover, provides a considerably larger surface area of frictional contact between the drape 10 and the patient's abdomen, pelvis and legs than known drapes. Importantly, the surface area of frictional contact between the drape 10 and the patient's abdomen, pelvis and legs generates substantial resistance to the drape 10 readily moving, sliding, falling off the patient, or otherwise be being easily displaced.

In various implementations, the pleats 22 are fabricated from the same material as the main body 14. Or, alternatively, the pleats 22 can be fabricated for a different material, for example, a mesh material or an elastic or stretchable material. The pleats 22 can be connected to the main body 14 in any suitable fashion, for example, the pleats can be ultrasonically welded, sewn or glued to the main body 14.

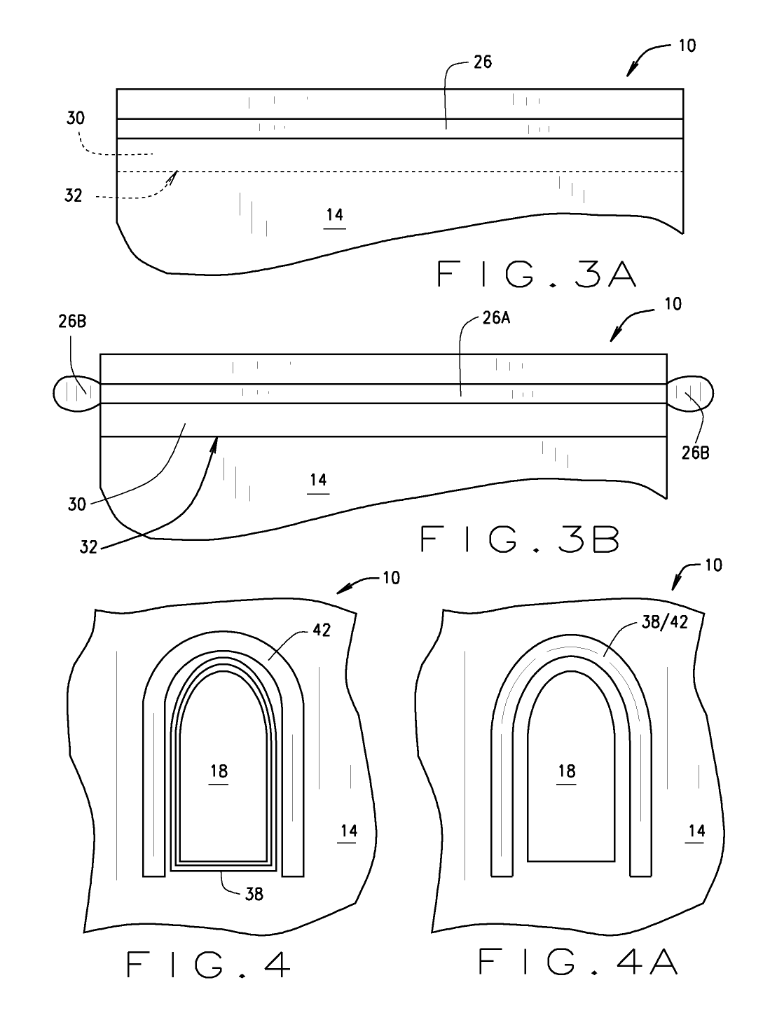

Referring now to FIGS. 1, 3A and 3B, as described above, in various embodiments, the drape 10 can include a drape retention device 26 that is structured and operable to wrap and contour the drape 10 around the patient's abdomen, pelvis and legs, and to retain the disposition and placement of the drape 10 on the patient so that the drape will not readily move, slide, fall off the patient, or otherwise be easily displaced.

With particular reference to FIG. 3A, in various implementations, the drape retention device 26 can comprise an adhesive strip disposed on the back side of the top portion of the drape 10, i.e., the top portion of the main body 14. In such implementations, the adhesive stripe 26 is structured and operable to removably adhere the top portion of the drape 10 to the patient's abdomen such that the drape 10 is held in place and will not readily move, slide, fall off the patient, or otherwise be easily displaced. It is envisioned that the adhesive strip 26 can be any suitable surgical/medical adhesive material that is sprayed, brushed, rolled or otherwise disposed directly onto the back side of the drape 10, i.e., main body 14. Alternatively, the adhesive strip 26 can be a strip of double-sided tape disposed on the back side of the drape 10. Additionally, a strip of removable non-adhesive backing material, e.g., waxed or coated paper, can be placed over the adhesive strip 26 to preserve the viability of the adhesive strip 26 until deployment of the drape 10 on the patient, at which time the non-adhesive backing can be removed to expose the adhesive strip 26, whereafter the adhesive strip 26 can be pushed into contact with the patient's abdomen.

With particular reference to FIG. 3B, in various implementations, the drape retention device 26 can comprise a strap and weight system comprising a strap 26A disposed across a front side of the disposition flap 30 and a pair of weights 26B, e.g., small sandbags, small beanbags or plastic or metal strip weights, connected to the opposing ends of the strap 26A. The strap 26A can be any device suitable for connecting the weights 26B across the width of the drape 10. For example, in various implementations, it is envisioned that the strap 26A can be an elastic or non-elastic strap, strips, string, strings, etc., of suitably strong and sturdy material attached to or integrally formed with the disposition flap 30 having the weights 26B connected thereto at opposing ends. In such embodiments, the top portion of the drape 10 is positioned across the patient's abdomen such that the weights 26B hang from patient's sides, thereby pulling the top portion of the drape 10 taunt and firmly across the patient's abdomen such that the drape 10 is held in place and will not readily move, slide, fall off the patient, or otherwise be easily displaced. Alternatively, the weights 26B can be separate and independent from the strap 26A and be removable connectable, via clips, hooks, pins, snap, buttons, hook and loop material, etc. to the strap 26A.

Referring again to FIGS. 1, 3A and 3B, in various other embodiments, the drape retention device 26 can comprise the combination of the adhesive strip disposed on the back side of the top portion of the drape 10 and the strap and weight system disposed across a front side of the disposition flap 30.

Referring now to FIGS. 1 and 2, as described above, in various embodiments, the drape 10 can include the disposition flap 30 that forms the elongated pocket 32 along the top edge portion, wherein the pocket 32 is structured and operable to provide a sterile means for the physician (or other medical team member) to handle, dispose and position the drape 10 on the patient. Particularly, when deploying and positioning the drape 10 on the patient, the physician (or other medical team member) can slide their hands into the pocket 32 and thereby deploy and position the drape 10 as desired. More particularly, by deploying and positioning the drape 10 utilizing the pocket 32, the physician's (or other medical team member's) hands do not contact the unsterile skin of the patient. Additionally, when the physician's (or other medical team member's) hands are placed within the pocket 32 to deploy and position the drape 10, disposition flap 30 will cover the physician's (or other medical team member's) hands such that the patient cannot instinctively grab, and hence contaminate, the physician's (or other medical team member's) hand(s).

Referring now to FIGS. 1, 2, 4 and 4A, as described above, in various embodiments, the drape 10 can include a reinforcement frame 38 disposed on or integrally formed with the main body 14 around at least a portion of the access opening 18. For modesty, dignity and discretion purposes, the interior of the access opening 18 has been concealed in FIG. 2. The reinforcement frame 38 is structured and operable to provide integrity and support to the access opening 18 such that the access opening 18 will remain open and not collapse or otherwise close during use of the drape 10, and hence, will not interfere with the physician's access to the patient's vaginal area during the respective surgical procedure. It is envisioned that the reinforcement frame 38 can be disposed on or integrally formed with the main body 14 around at least a portion of the access opening 18 on the front side of the drape 10, the back side of the main body 14 or both. However, FIG. 4 exemplarily illustrates the reinforcement frame 38 as being disposed on or integrally formed with the main body 14 around the entire access opening 18 on the back side of the main body 14.

The reinforcement frame 38 can be comprised of any material suitable to provide integrity and support to the access opening 18. For example, in various embodiments, the reinforcement frame 38 can be formed by ultrasonic welding one or more semi-stiff or semi-rigid ridges or beads, e.g., a plurality of ridges or beads disposed in a crisscross pattern, around at least a portion of the periphery of the access opening 18, or in close proximity thereto. Ultrasonic welding is commonly used to bond or connect together portions of known surgical drapes, covers, gowns, hats, shoe covers, etc. Such ultrasonic welding typically creates a thickened area at the weld sight, whereby the thickened weld has greater stiffness or rigidity than the base material adjacent the weld sight and comprising the remaining portion of the respective surgical drape, cover, gown, hat, shoe cover, etc. Alternatively, in various other embodiments, the reinforcement frame 38 can comprise one or more metal, plastic or foam wires or strips that are bonded to or integrally formed with the main body 14 around at least a portion of the access opening 18.

In addition to providing integrity and support to the access opening 18, in various embodiments, the reinforcement frame 38 can be comprised of a malleable material, e.g., one or more malleable metal wires or strips, such that the malleable reinforcement frame 38, and hence the drape main body 14, and access opening 18, can be formed by the physician (or other medical team member) to have a contour that is similar to the shape of the patient's body adjacent the access opening 18. Accordingly, by contouring the the access opening 18 to the shape of the patient's body, the access opening 18 and the portion of the main body 14 surrounding the access opening 18 will be less likely to interfere with access to the vaginal area by the physician during the respective surgical procedure.

As also described above, in various embodiments, the drape 10 can further include at least one adhesive strip 42 disposed on the back side of the main body 14 around at least a portion of the access opening 18 periphery, or in close proximity thereto. The adhesive strip(s) 42 is/are structured and operable to removably adhere the main body 14, particularly the periphery of the access opening 18 (or the portion of the main body 14 in close proximity to opening periphery), to the patient so that the access opening 18 and the portion of the main body 14 surrounding the access opening 18 will not interfere with the physician's access to the vaginal area during the respective surgical procedure. It is envisioned that the adhesive strip(s) 42 can be any suitable surgical/medical adhesive material that is sprayed, brushed, rolled or otherwise disposed directly onto the back side of the main body 14 around the access opening 18. Alternatively, the adhesive strip(s) 42 can be one or more strips of double-sided tape disposed on the back side of the main body 14 around the access opening 18. Additionally, one or more strips of removable non-adhesive backing material, e.g., waxed or coated paper, can be placed over the adhesive strip(s) 42 to preserve the viability of the adhesive strip(s) 42 until deployment of the drape 10 on the patient, at which time the non-adhesive backing can be removed to expose the adhesive strip(s) 42, whereafter the adhesive strip(s) 42 can be pushed into contact with the patient's skin.

Furthermore, in various embodiments, the reinforcement frame 38 and the adhesive stripes 42 can be the one-and-the-same to provide an adhesive reinforcement frame 38/42, as illustrated in FIG. 4A. For example, in various embodiments, the adhesive reinforcement frame 38/42 can comprise double-sided foam or cardboard tape disposed on the back side of the main body 14 in a horseshoe shaped around a portion of the access opening 18. In such embodiments, the adhesive reinforcement frame 38/42 would provide both structural rigidity or stiffness around the access opening 18 and the ability removably adhere the periphery of the access opening 18 (or the portion of the main body 14 in close proximity to opening periphery), to the patient. Accordingly, in such embodiments the adhesive reinforcement frame 38/42 will provide integrity and support to the access opening 18, and securability of the main body 14 around the opening 18 to the patient, such that the access opening 18 will remain open and not collapse or otherwise close during use of the drape 10, and hence, will not interfere with the physician's access to the patient's vaginal area during the respective surgical procedure.

Referring now to FIGS. 1 and 2, as described above, in various embodiments, the drape 10 can include the plurality of equipment retention devices 34 that are attached to or integrally formed with the drape main body 14, and are structured and operable to provide the respective physician with multiple means for the placement and retention of various types of surgical equipment, e.g., forceps, hemostats, sutures, gauze, sponges, and other surgical instruments, tools and supplies, during the respective procedure. The equipment retention devices 34 are formed, connected to and/or disposed on the drape main body 14, and strategically located on the main body 14 based on the purpose, function, frequency of use and desired ergonomic convenience of the respective retention device 34. The retention devices 34 include various different devices, e.g., loops, tabs, hooks, pockets, clips, clamps, clasps, blocks, etc., wherein each respective retention device 34 is structured and operable to provide a respective function.

For example, the retention devices 34 can include one or more pockets or pouches 34A that are structured and operable to hold such surgical supplies as surgical gauze, swabs, sponges, etc. Each pocket 34A has an open top, that in various embodiments can be covered by a flap, suitable for allowing placement of the respective surgical gauze, swabs, sponges, etc., into the pocket 34A. Additionally, in various embodiments, each pocket 34A can include a dispensing opening 46 disposed in a side or bottom of the pocket 34A for convenient dispensing of such surgical gauze, swabs or sponges. The retention devices 34 can additionally include one or more instrument or tool loops 34B that are structured and operable to hold or have clipped thereto various surgical instruments or tools such as forceps, hemostats, scissors, tweezers, etc. For example, forceps and hemostats can be clipped to the instrument loops 34B, while tweezers and scissors can be inserted through the instrument loops 34B, as exemplarily illustrated in FIG. 2. The instrument loops 34B can be fabricated from any suitable material. For example, in various embodiments, the instrument loops 34B are fabricated from the same material as the main body 14. Alternatively, in various embodiments, the instrument loops 34B can be fabricated from nylon, plastic or an elastic material.

Furthermore, the retention devices 34 can include clips, hooks, clamps or clasps 34C that are structured and operable to hold or have hung therefrom various surgical instruments and/or tools such as forceps, hemostats, scissors, tweezers, etc. In various embodiments the clips 34C can comprise snap-clips that are structured and operable to automatically close a set of opposing jaws around the respective instrument/tool when the instrument/tool is placed into to open jaws. Such snap-clips are further structured and operable to easily open the jaws to release the respective instrument/tool when the respective instrument/tool is gently pulled from the respective snap-clip. Additionally, the retention devices 34 can include one or more needle retention blocks 34D that are structured and operable to retain suture needles that are inserted into the needle retention block(s) 34D. In various embodiments, the needle retention block(s) 34D is/are constructed to be cubical and of a foam type material into which suture needles can be inserted. However, it is envisioned that the needle retention block(s) 34D can have any shape, e.g., cylindrical, conical, spherical, pyramidal, etc., and be constructed of any other material suitable to receive and retain suture needles and remain within the scope of the present disclosure.

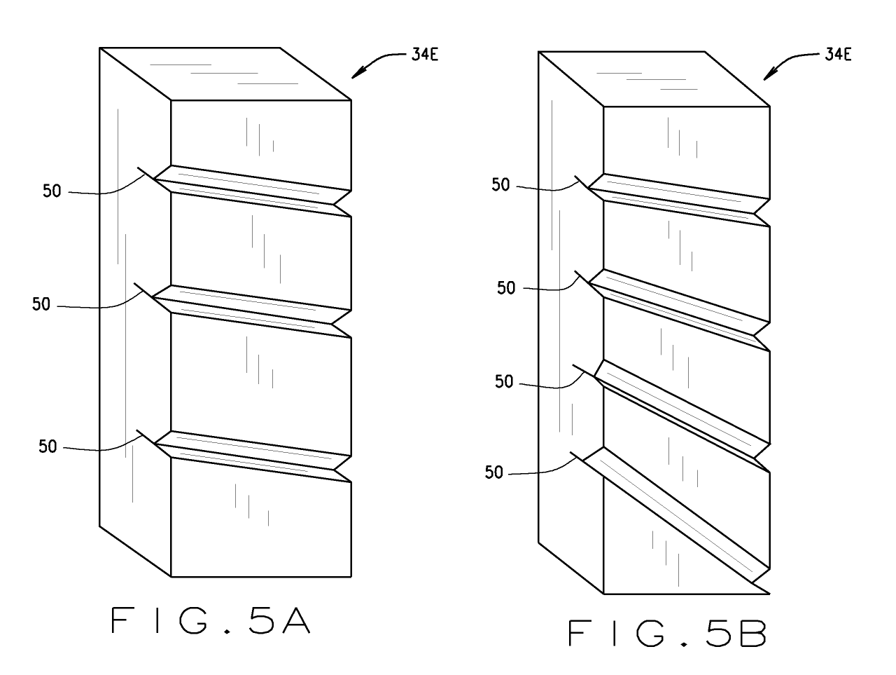

Referring now to FIGS. 1, 2 5A and 5B, in various embodiments, the retention devices 34 can further include one or more suture thread retraction blocks 34E. The suture thread retraction block(s) 34E is/are disposed on the drape main body 14 adjacent the side(s) of the access opening 18. The suture thread retraction block(s) 34E is/are structured and operable to retain suture thread, particularly suture thread that extends from a suture knot, e.g., lengths of the suture thread that has been used to dispose a suture in the patient, but has not yet been cut free from the respective suture knot. Each suture thread retraction block 34E is constructed of a foam or sponge-like material and includes a plurality of slits 50 disposed in a face of the retraction block 34E. During the respective procedure, the physician can utilize the retraction block(s) to 34E to temporarily hold the suture thread by easily pushing the suture thread within one of the slits 50. Consequently, the foam or sponge-like material of the retraction block(s) 34E will gently compress against the suture thread and retract the suture thread, i.e., hold the suture thread off to the side of the access opening 18, until such time as the physician gently and easily pulls the suture thread out of the slit 50. Although the suture thread retraction block(s) 34E is/are illustrated as having the shape of right rectangular prism, it is envisioned that the retraction block(s) 34D can have any shape, e.g., a right triangular prism, and remain within the scope of the present disclosure. Additionally, the slits 50 can be disposed along the length of the respective retraction block 34D in any desirable arrangement, spacing and orientation. For example, as illustrated in FIG. 5A, the slits 50 can be disposed in a parallel angled arrangement along the length of the retraction block 34D, wherein each slit 50 is angled toward a bottom of the access opening at the same angle, e.g., 15° to 35°. Alternatively, as illustrated in FIG. 5B, in various other embodiments, the slits 50 can be disposed in a nonparallel angled arrangement along the length of the retraction block 34D, wherein the slits 50 are angled toward the bottom of the access opening at various different angles.

In summary, the a surgical drape 10, as described herein, provides a sterile field around the vaginal area of a patient during obstetrical laceration repair procedures, episiotomy repair procedures following vaginal delivery, and other vaginal surgery procedures. Additionally, the drape 10, as described herein, is structured and operable to provide the physician with multiple means for the convenient placement and retention of various types of surgical equipment during the respective procedure, via the equipment retention devices 34. Moreover, the drape 10, as described herein, is structured and operable to be disposed and positioned on the patient such that the drape 10 can be contoured to the patient's body, via the pleats 22 and malleable reinforcement frame 38, and retained in place, via the drape retention device 26, the adhesive strip(s) 42, and the contoured fit of the drape 10, such that will not readily move, slide, fall off, or otherwise be easily displaced, even with the various types of surgical equipment, e.g., forceps, hemostats, sutures, gauze, sponges, and other surgical instruments, tools and supplies, connected to and/or retained on the drape 10, via the retention device 34, as described above.

The description herein is merely exemplary in nature and, thus, variations that do not depart from the gist of that which is described are intended to be within the scope of the teachings. Such variations are not to be regarded as a departure from the spirit and scope of the teachings.

What is claimed is:

1. A surgical drape for providing a sterile field around the vaginal area of a patient during vaginal surgical procedures, said drape comprising:
a main body;
an access opening centrally disposed within the main body;
a plurality of pleats disposed along opposing lateral side portions of main body, the pleats structured and operable to:
allow the drape to be contoured around the patient's abdomen, pelvis and legs; and
provide stability to the drape such that the drape will not be easily displaced from the patient; and
a plurality of equipment retention devices disposed on the main body that are structured and operable to removably retain surgical equipment during the respective surgical procedure.

2. The drape of claim 1 further comprising a drape retention device disposed along a top portion of the drape, the drape retention devices structured and operable to retain the disposition of the drape on the patient so that the drape will not be easily displaced from the patient.

3. The drape of claim 2, wherein the drape retention device comprises an adhesive strip disposed on a back side of the top portion of the drape, the adhesive strip structured and operable to removably adhere to the patient's abdomen such that the drape will not be easily displaced from the patient.

4. The drape of claim 1 further comprising a disposition pocket formed along a top portion of the drape, the disposition pocket structured and operable to provide a sterile means for disposition of the drape on the patient.

5. The drape of claim 1 further comprising a reinforcement frame disposed on the main body around at least a portion of the access opening, the reinforcement frame structured and operable to provide support to the access opening such that the access opening will remain open during the respective surgical procedure.

6. The drape of claim 5, wherein the reinforcement frame comprises a malleable material that is structured and operable to be formed by the physician to have a contour that is similar to the shape of the patient's body adjacent the access opening.

7. The drape of claim 1 further comprising at least one adhesive strip disposed on the back side of the main body around at least a portion of the access opening, the at least one adhesive strip structured and operable to removably adhere the access opening to the patient such that the access opening will not interfere with the physician's access to the vaginal area during the respective surgical procedure.

8. The drape of claim 1, wherein at least one of the equipment retention devices comprises a suture thread retraction block disposed on the main body adjacent the access opening, the suture thread retraction block structured and operable to selectively removably retain suture thread during the respective surgical procedure.

9. A surgical drape for providing a sterile field around the vaginal area of a patient during vaginal surgical procedures, said drape comprising:
a main body;
an access opening centrally disposed within the main body;
a plurality of pleats disposed along opposing lateral side portions of main body, the pleats structured and operable to:
allow the drape to be contoured around the patient's abdomen, pelvis and legs; and
provide stability to the drape such that the drape will not be easily displaced from the patient; and
a plurality of equipment retention devices disposed on the main body that are structured and operable to removably retain surgical equipment during the respective surgical procedure, wherein at least one of the equipment retention devices comprises a suture thread retraction block disposed on the main body adjacent the access opening, the suture thread retraction block structured and operable to selectively removably retain suture thread during the respective surgical procedure.

10. The drape of claim 9 further comprising a drape retention device disposed along a top portion of the drape, the drape retention devices structured and operable to retain the disposition of the drape on the patient so that the drape will not be easily displaced from the patient.

11. The drape of claim 10, wherein the drape retention device comprises an adhesive strip disposed on a back side of the top portion of the drape, the adhesive strip structured and operable to removably adhere to the patient's abdomen such that the drape will not be easily displaced from the patient.

12. The drape of claim 9 further comprising a disposition pocket formed along a top portion of the drape, the disposition pocket structured and operable to provide a sterile means for disposition of the drape on the patient.

13. The drape of claim 9 further comprising a reinforcement frame disposed on the main body around at least a portion of the access opening, the reinforcement frame structured and operable to provide support to the access opening such that the access opening will remain open during the respective surgical procedure.

14. The drape of claim 13, wherein the reinforcement frame comprises a malleable material that is structured and operable to be formed by the physician to have a contour that is similar to the shape of the patient's body adjacent the access opening.

15. The drape of claim 9 further comprising at least one adhesive strip disposed on the back side of the main body around at least a portion of the access opening, the at least one adhesive strip structured and operable to removably adhere the access opening to the patient such that the access opening will not interfere with the physician's access to the vaginal area during the respective surgical procedure.

16. A surgical drape for providing a sterile field around the vaginal area of a patient during vaginal surgical procedures, said drape comprising:
a main body;
an access opening centrally disposed within the main body;
an adhesive reinforcement frame disposed on the main body around at least a portion of the access opening, the adhesive reinforcement frame structured and operable to provide support to the access opening and to removably adhere the of the access opening to the patient such that the access opening will remain open and will not interfere with the physician's access to the vaginal area during the respective surgical procedure;
a plurality of pleats disposed along opposing lateral side portions of main body, the pleats structured and operable to:
allow the drape to be contoured around the patient's abdomen, pelvis and legs; and
provide stability to the drape such that the drape will not be easily displaced from the patient; and
a plurality of equipment retention devices disposed on the main body that are structured and operable to removably retain surgical equipment during the respective surgical procedure, wherein at least one of the equipment retention devices comprises a suture thread retraction block disposed on the main body adjacent the access opening, the suture thread retraction block structured and operable to selectively removably retain suture thread during the respective surgical procedure.

17. The drape of claim 16 further comprising a drape retention device disposed along a top portion of the drape, the drape retention devices structured and operable to retain the disposition of the drape on the patient so that the drape will not be easily displaced from the patient.

18. The drape of claim 17, wherein the drape retention device comprises an adhesive strip disposed on a back side of the top portion of the drape, the adhesive strip structured and operable to removably adhere to the patient's abdomen such that the drape will not be easily displaced from the patient.

19. The drape of claim 16 further comprising a disposition pocket formed along a top portion of the drape, the disposition pocket structured and operable to provide a sterile means for disposition of the drape on the patient.

20. The drape of claim 16, wherein the reinforcement frame comprises a malleable material that is structured and operable to be formed by the physician to have a contour that is similar to the shape of the patient's body adjacent the access opening.
